(19) United States
(12) Patent Application Publication
(10) Pub. No.: US 2005/0165491 A1
(43) Pub. Date: Jul. 28, 2005

(54) Method and apparatus for bi-compartmental partial knee replacement

(76) Inventor: Robert L. Diaz, Palm Beach Gardens, FL (US)

Correspondence Address: MCHALE & SLAVIN, P.A., 2855 PGA BLVD, PALM BEACH GARDENS, FL, 33410, US

(21) Appl. No.: 10/769,478

(22) Filed: Jan. 23, 2004

(51) Int. Cl. A61F 2/38

(52) U.S. Cl. 623/20.19; 623/20.3; 623/20.35

(57) ABSTRACT

A partial knee replacement involving two compartments of the knee includes the installation of a femoral prosthesis shaped to protect two contiguous compartments of the knee, namely the Trochlea and the Condyle. A Tibial prosthesis protects the tibial surface.

### FIELD OF THE INVENTION

This invention relates to orthopedic surgery, particularly to minimally invasive bi-compartmental partial knee replacement, inclusive of instrumentation and methods therefore.

### BACKGROUND OF THE INVENTION

Total knee replacement is a conventional surgical approach for treatment of trauma and disease of the knee involving more than one of the three compartments of the joint. The traditional total knee replacement starts with a 10 to 12 inch incision over the knee, the quadriceps muscle is split or moved to one side disrupting the patella, the ends of the femur and tibia are re-shaped removing all the cartilage, and matching femoral and tibial implants are driven into the shaped ends. Polyethylene inserts in the tibial implant articulates with the polished femoral component and replaced cartilage. The patella is reshaped and a prosthesis added to smoothly slide over the femoral implant. The muscle, tendons and ligaments are reconstructed to complete the surgery. Usually, there is a hospital stay of 3-5 days and an intensive rehabilitation period of up to 3 months follows. Most of this post-operative schedule is for regaining movement due to the trauma to the muscles, tendons and other soft tissue.

In some cases, only one compartment of the knee joint is affected by trauma or disease, e.g., a medial condyle of the femur and medial plateau of the tibia. In this situation, a partial knee replacement (uni-compartmental) is done in which only the affected portion of the joint is removed and replaced with a prosthesis, such as taught by Treace, U.S. Pat. No. 4,193,140 issued Mar. 18, 1980. The operation may be completed with a much smaller incision, e.g., 3-5 inches on one side of the knee, and only a portion of the cartilage need be removed. The surgical trauma is much less extensive and the recovery time is greatly shortened.

There are conventional surgical procedures for reconstruction of the patella or kneecap, in the event only the patella-femoral compartment is distressed. Colleran et al, U.S. Pat. No. 5,944,723, and Petersen, U.S. Pat. No. 4,633,862, both teach the use of a forceps-like instrument to grasp the patella for reshaping, as part of a uni-compartmental operation or a total knee replacement.

However, the common practice if more than one compartment is involved is to do a total knee replacement. The general theory being that if more than one compartment is affected, it indicates a broader deteriorating condition and a partial knee replacement would be merely postponing the inevitable total replacement. Whether or not this is true, the result of such a decision is to require the patient to endure pain, long term rehabilitation, and diminished quality of life that his immediate condition does not warrant. In the older patients, the degree of mobility may never reach the potential for a total knee replacement.

U.S. Pat. No. 4,034,418 to Jackson et al discloses the use of separate prostheses for a bi-compartmental, medial and lateral compartments.

What is needed in the art is a partial knee replacement that addresses two contiguous compartments of the knee without resorting to a total replacement thereby providing all the advantages of minimal surgical trauma and a short rehabilitation of conventional partial knee replacements.

### SUMMARY OF THE PRESENT INVENTION

Disclosed is a prosthesis for a bi-compartmental partial knee replacement. The prosthesis includes an integral trochlear extension adapted to encompass the trochlear of a femur in a contiguous compartment of the knee, and an arcuate femoral prosthesis for fitting about one condyle of the femur.

Therefore, it is an objective of this invention to teach the performance of the partial knee replacement including the prosthesis and surgical implements.

It is another objective of this invention to teach the performance of a partial knee replacement involving two compartments of the knee in which the patella-femoral is one.

It is a further objective of this invention to teach the use of a patella button with an extension of the unicondylar femoral component for partial knee replacement.

It is still another objective of this invention to use the femoral prosthesis and a tibial prosthesis to replace the diseased medial or lateral compartment, medial or lateral femoral condyle, and medial or lateral tibial plateau with the medial or lateral femoral prosthesis replacing the diseased patella-femoral (trochlea) compartment. Specifically, the femoral prosthesis replaces the medial or lateral femoral condyle and the diseased trochlea of the femur. The tibial prosthesis replaces the diseased medial or lateral tibial plateau. The patella prosthesis replaces the diseased surface of the patella.

### BRIEF DESCRIPTION OF THE DRAWINGS

FIG. 1 illustrates a side view, partially in section, of the knee and bi-compartmental prostheses of this invention;

FIG. 2 illustrates a front view of the knee prostheses of FIG. 1;

FIG. 3 illustrates a prospective of the femoral prosthesis of this invention;

FIG. 4 illustrates a prospective of the tibial prosthesis of this invention;

FIG. 5 illustrates a perspective of the bi-compartmental prosthesis in the flexed position showing the medial compartment, fibula and patella femoral (trochlear) compartment; and

FIG. 6 illustrates a perspective of the bi-compartmental prosthesis in the flexed position showing the lateral compartment, fibula and patella femoral (trochlear) compartment.

### DETAILED DESCRIPTION OF THE INVENTION

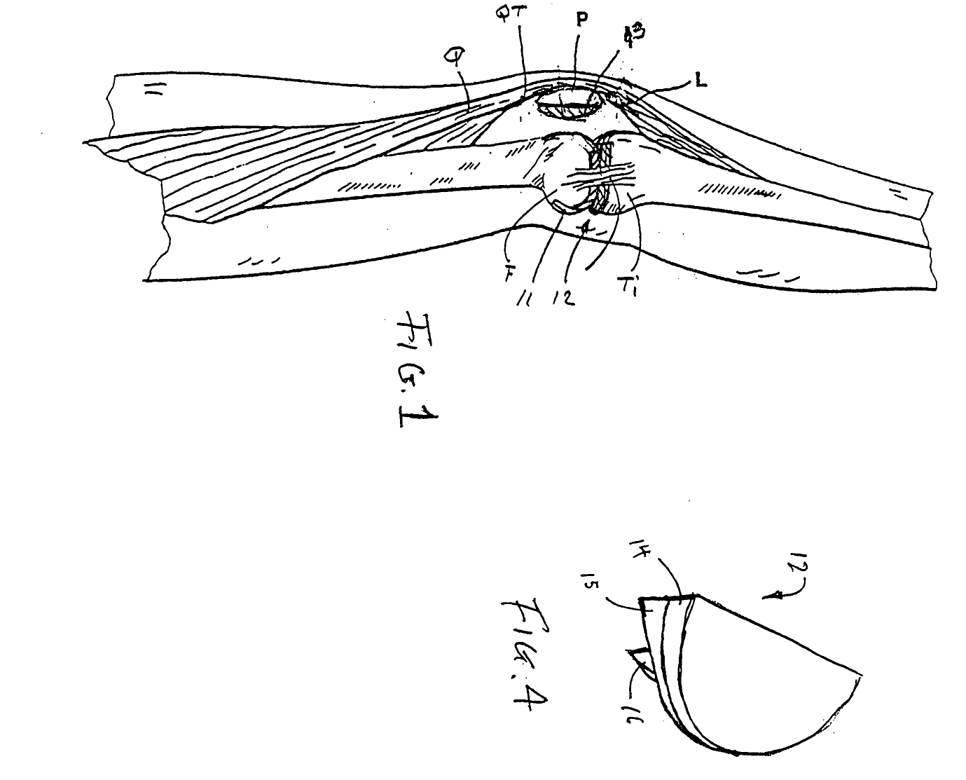

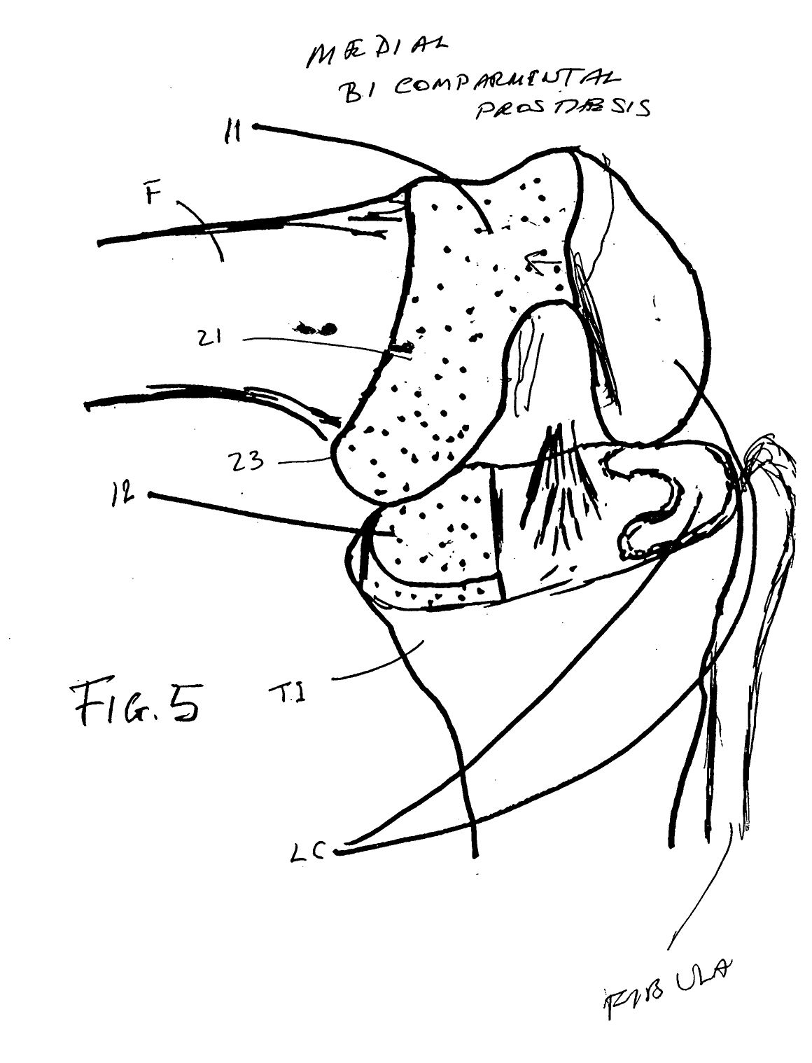

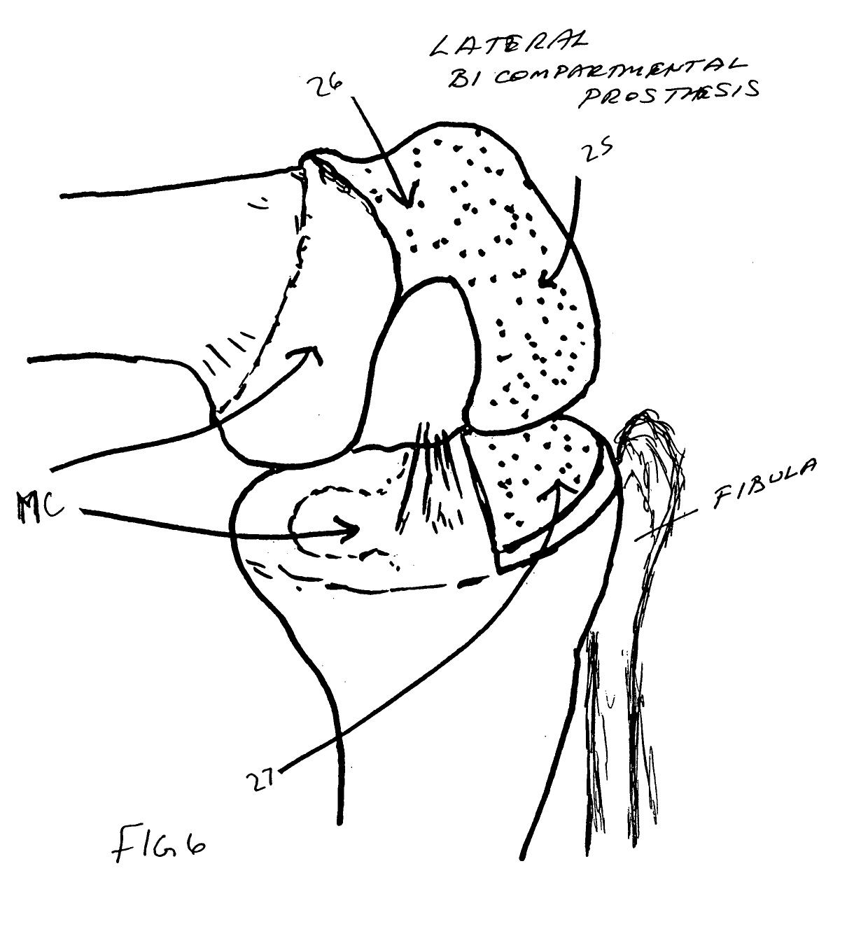

Referring to the figures in general, a patient's leg is illustrated with the medial compartment and the contiguous patello-femoral compartment exposed to show the prostheses attached to the medial condyle of the femur F, the proximal end of the tibia Ti, and the kneecap P. FIG. 5 depicts the remaining normal lateral compartment LC, including the lateral condyle of the femur and tibia is illustrated. Also shown in the FIG. 1 is the quadriceps muscle Q, the quadriceps tendon QT, and the patellar ligament L. The arcuate femoral prosthesis 11, shown in FIG. 3, is attached to the medial condyle, the tibial prosthesis 12, shown in FIG. 4, is attached to the tibia and the kneecap button 13 is attached to the patella as shown in FIG. 1. This is a result of an operation accessing the knee joint through a small incision in the medial aspect, (inside), of the knee. This same result may also be accomplished through the lateral compartment by an incision on the outside of the knee. The operation minimally reshapes one condyle of the femur, the trochlea and the medial aspect of the proximal end of the tibia but does not destroy the entire cartilage of the knee nor does it destroy the natural connection between the quadriceps and the knee.

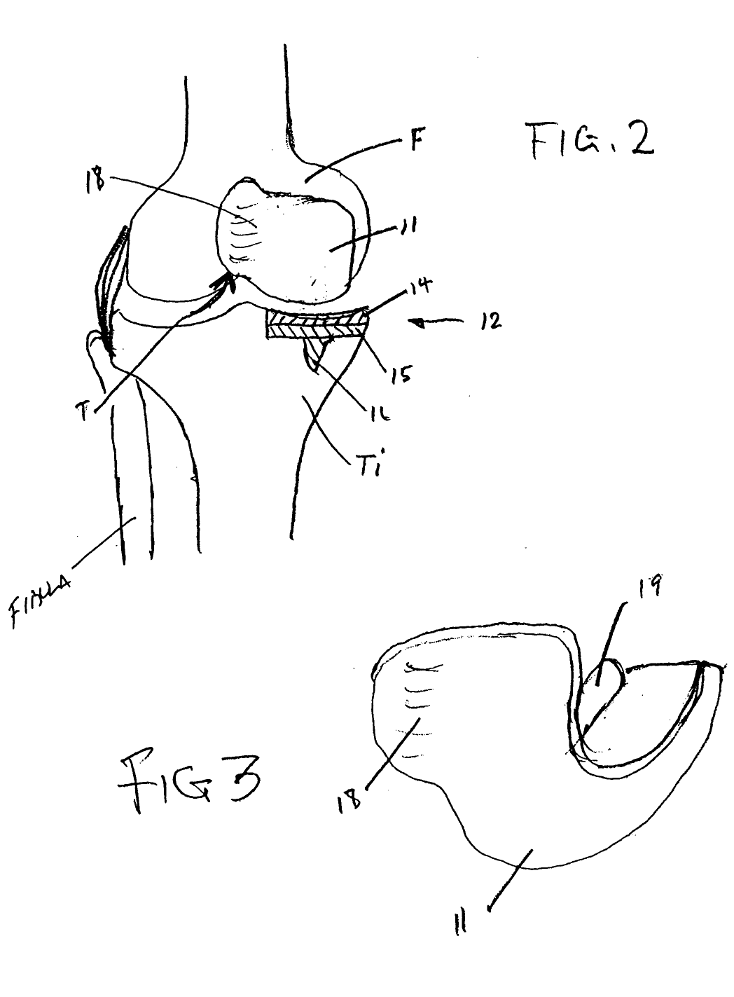

FIG. 2 shows the front or anterior view of the medial compartment of the knee including shown figula and the medial condyle F of the femur and the trochlea T. The femoral prosthesis 11 is arcuately shaped to closely fit around the distal end of the femur and has a trochlear extension 18 for replacing the surgically repaired surface of the trochlea T. The femoral prosthesis 11 has a femoral anchor 19 that is received in a groove or pilot hole formed in the condyle of the femur to secure the prosthesis to the bone, with or without cement. As shown, the anchor is in the form of a peg but could be shaped as an elongated blade. The femoral prosthesis may be made of conventional materials used for knee prostheses including magnetic metals, non-magnetic metals, ceramics, and polymers or combinations of any of these materials or any other materials with the requisite bio-compatible properties and strength. The external surface of the femoral prosthesis rides on the artificial material forming the proximal surface of the tibial pad.

The tibial prosthesis 12 has a tray 15 including a tibial anchor 16 to attach the prosthesis to the tibia Ti. The tray 15 has a proximal surface with an attached pad 14 which serves to replace the cartilage normally existing between the femur and the tibia and provide a smooth bearing surface contacting the femoral prosthesis 11. The pad 14 may be removably attached to the tray 15 in the event that wear, over time, requires replacement.

During the operation, the patella P shown in FIG. 1 may be turned or rotated only to the extent necessary to establish a cutting plane across the dorsal surface for reshaping the patella to accept the prosthetic button without permanently disturbing the tendons and ligaments. This is accomplished through the natural resilience of the tissues. In some cases, the trochlea T, only, may be surgically repaired and the patella P may not require surgical intervention. The button 13 may be a ceramic, polymer, such as polyethylene, or the like which provides a smooth, slick surface to interact with the trochlear extension 18. A retractor having a dish shaped end, not shown, may be inserted between the patella P and the femur F and pivoted upwardly or anteriorly seating the patella in the dish shaped depression because of the resistance of the quadriceps Q. The dish shaped depression may have a cutting slot in the wall or have an open bottom. The posterior or ventral or articular surface of the patella extending below the slot or bottom can be shaved with a reciprocating blade in a plane along the surface of the retractor. Other instruments may be used, in the nature of forceps, to manipulate the patella and provide a cutting guide.

Once the posterior, ventral or articular surface of the patella P is reshaped, a drill guide is placed against the surface to establish the location of bores in the patella matching the location of pegs formed on the button 13. These bores may be angled or perpendicular to the dorsal surface of the patella. The angled bores permit insertion of the button 13 in less vertical space. The drill guide also serves as a stop for the drill bit to prevent undue penetration of the patella. The button is attached to the patella with the pegs placed in the bores using cement, if desired. The small incision on the side of the knee is then closed to complete the operation.

Again referring to the figures set forth is a perspective of the bi-compartmental prosthesis in the flexed position showing the medial compartment 21 and patella femoral (trochlear) compartment 11, the remaining normal knee is the lateral compartment LC. In this embodiment, the medial tibial prosthesis 12 is positioned in operation with the medial compartment and patella femoral compartment shown in general as numeral 23.

FIG. 6 sets forth a perspective of the bi-compartmental prosthesis in the flexed position showing the lateral compartment 25 and patella femoral (trochelea) compartment 26, the remaining normal knee is the medial compartment MC. The trochlear compartment 26 and lateral condyle compartment 25 provide this prostheses with the lateral tibial prosthesis 27 for engagement of the lateral condyle.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A prosthesis for a bi-compartmental partial knee replacement consisting of an arcuate femoral prosthesis for fitting about one condyle of the femur in a compartment of the knee, a bone anchor connected to said femoral prosthesis for securing said femoral prosthesis to a femur, said femoral prosthesis having an integral trochlear extension adapted to cover the trochlear of a femur in a contiguous compartment of the knee, whereby said femoral prosthesis provides a trochlear prosthesis and a condyle for articulating with a tibial prosthesis.

2. A prosthesis of claim 1 further comprising said femoral prosthesis has an elongated narrow body arcuate from end to end, said trochlear extension formed on one end portion of said body.

3. A prosthesis of claim 1 further consisting of a tibial prosthesis for attachment to the shaped proximal surface of the tibia in one compartment of the knee, said tibial prosthesis providing a bearing surface for said femoral prosthesis.

4. A prosthesis of claim 3 further comprising a patella button for attachment to the shaped dorsal surface of the patella, said button providing a bearing surface between the patella and said trochlear extension of said femoral prosthesis.

5. A prosthesis of claim 2 further comprising a tibial prosthesis for attachment to the shaped proximal surface of tibia in one compartment of the knee, said tibial prosthesis having a tray, said tray having an anchor for insertion into the proximal surface of the tibia, said tray adapted to secure a pad providing a bearing surface for the femoral prosthesis.

6. A prosthesis of claim 5 further comprising a patella button for attachment to the shaped articulating, ventral or posterior surface of the patella, said button providing a bearing surface between the patella and said trochlear extension of said femoral prosthesis.

7. A prosthesis of claim 2 further comprising a patella button for attachment to the shaped dorsal surface of the patella, said button providing a bearing surface between the patella and said trochlear extension of said femoral prosthesis.

8. A prosthesis of claim 3 further comprising said bearing surface of said tibial prosthesis being concave.

9. A device for bicompartmental arthoplasty of the knee comprising:
a monolithically formed medial, but not lateral, femoral prosthesis component having a first internal surface configured to be secured to a surgically prepared medial compartment of a distal end of a patient's femur and a second exterior convex curved surface positioned and configured to replicate a medial femoral condyle, said medial femoral prosthesis component also having a concave trochlea surface positioned and configured to articulate with the patella; and
a unicondyle medial tibial prosthesis component having a first interior surface configured to be secured to a surgically prepared medial compartment of a proximal end of the patient's tibia and a second concave curved exterior surface configured to receive the second convex curved surface of said medial femoral prosthesis component to permit pivotal articulation between said medial femoral prosthesis component and said medial tibial prosthesis component, said pivotal articulation approximating the articulation of a healthy knee joint.

10. A device for bicompartmental arthoplasty of the knee comprising:
a monolithically formed medial, but not lateral, femoral prosthesis component having a first leg portion and a second leg portion and a base portion connected between said first and said second leg portions to form an essentially U shaped component, said medial femoral prosthesis component having a first interior surface comprising a base interior surface, an anterior interior surface and a posterior interior surface, said anterior interior surface and said posterior interior surfaces being essentially parallel to one another and being configured to be secured to a surgically prepared medial compartment of a distal end of the patient's femur so that the base interior surface contacts the distal end of the femur, said anterior interior surface contacts an anterior surface of the distal end of the femur, and the posterior interior surface contacts a posterior surface of the distal end of the femur, and said medial prosthesis component also having a second exterior convex curved surface on said base portion and configured to replicate a medial femoral condyle, and a concave trochlea surface positioned on said first leg portion and configured to articulate with the patella; and
a unicondyle medial tibial prosthesis component having a first surface configured to be secured to a surgically prepared medial compartment of the proximal end of the tibia and a second concave curved surface configured to receive the second convex curved surface of said medial femoral prosthesis component to permit pivotal motion between said medial femoral prosthesis component and said medial tibial prosthesis component, said pivotal motion approximating the motion of a healthy knee joint.

11. A device for bicompartmental arthoplasty of the knee comrpising:
a monolithically formed medial, but not lateral, femoral prosthesis component having a first internal surface configured to have at least one flat surface to be secured to a surgically prepared medial compartment of a distal end of a patient's femur and a second exterior convex curved surface positioned and configured to replicate a medial femoral condyle, said medial femoral prosthesis component also having a concave trochlea surface positioned and configured to articulate with the patella, and
a unicondyle medial tibial prosthesis component having a first interior surface configured to be secured to a surgically prepared medial compartment of a proximal end of the patient's tibia and a second concave curved exterior surface configured to receive the second convex curved surface of said medial femoral prosthesis component to permit pivotal articulation between said medial femoral prosthesis and said medial tibial prosthesis component, said pivotal articulation approximating the articulation of a healthy knee joint.
